(19) United States
(12) Patent Application Publication
(10) Pub. No.: US 2003/0180251 A1
(43) Pub. Date: Sep. 25, 2003

(54) Medical technical product, method for producing the same and providing the same for surgery

(76) Inventors: Volker Friedrich, Tuttlingen (DE); Erich K Odermatt, Schaffhausen (CH); Christine Weis, Tuttlingen (DE)

Correspondence Address: NATH & ASSOCIATES, 1030 15th STREET, 6TH FLOOR, WASHINGTON, DC, 20005, US

(21) Appl. No.: 10/343,200

(22) PCT Filed: Jul. 28, 2001
(86) PCT No.: PCT/EP01/08768

(30) Foreign Application Priority Data
Aug. 2, 2000 (DE) 10037601.0
Apr. 6, 2001 (DE) 10117099.8

(51) Int. Cl. A61K 31/74

(52) U.S. Cl. 424/78.18

(57) ABSTRACT

A medicotechnical product for adhesion prophylaxis for the post-operative prevention of accretions in the body comprises at least one PVA (polyvinyl alcohol) selected from the group comprising uncrosslinked PVA with a molecular weight of 15,000 to 400,000, crosslinked PVA and mixtures thereof. The molecular weight of the PVA or the mixture is selected in such a way that it can be excreted via the kidneys substantially with no degradation of the PVA molecules.

The present invention relates to a medicotechnical product for adhesion prophylaxis for post-operative prevention of accretions in the body, a method for its manufacture and its provision for surgery.

The risk of undesired scarring arises during any surgery in the abdominal cavity of a patient. The operation represents a traumatization of the peritoneum, which leads to an inflammatory reaction with exudation of fibrin. The resulting accretions can be dissolved if there is adequate fibrinolytic activity within the abdominal cavity, so that there are no lasting complaints for the patient. However, if the enzymatic reaction in a patient is not adequate for lysing the fibrin deposits, fixed, permanent adhesions arise, which can lead to pain, delayed wound healing or malfunctions of abdominal organs in the patient in question.

Examples are general surgery and also laparoscopies in the gynecological field, where accretions are the frequent cause of pains in the lower abdomen and infertility.

Over the last few years increasingly re-operations, the replacement of artificial valves or the fitting of aortocoronary bypasses have been carried out in cardiac surgery. Pericardial adhesions caused during initial surgery represent a considerable risk for the patient with a high mortality rate due to bleeding complications or aortocoronary bypass injuries.

Peritoneal adhesion can occur in nephrology when carrying out continuous, ambulatory peritoneal dialysis. With accretions in the intestinal region frequently passage disturbances are observed.

Apart from the subjective ill feeling of the patient, such complications lead to extended medical treatment, chronic pain and increased cost.

By improving operative measures and surgery, the medical sector has long attempted to prevent accretions and the complications associated therewith. However, these have not been adequate, so that effective, additive measures are needed for prophylaxis of post-operative adhesions.

For the prophylaxis of peritoneal adhesions a number of substances have been proposed, such as e.g. the vitreous humor of calf eyes or olive oil. Although anticoagulants and fibrinolytics can bring about a reduction of accretions, the risk of post-operative bleeding has prevented widespread use in clinics. The local application of antibiotics has preponderantly led to an increase in adhesion formation. Most known adhesion prophylaxis substances are used with the aim of separating lesions of the peritoneum during healing from neighbouring tissues.

The problem of the invention is to provide a medicotechnical product for use in surgery, particularly for preventing undesired adhesion, which overcomes the difficulties of prior art medicotechnical products and which can be easily and inexpensively manufactured, whilst being easy to handle in practice using standard surgical methods.

The problem is solved by a medicotechnical product for adhesion prophylaxis for post-operative prevention of accretions in the body comprising at least one PVA (polyvinyl alcohol) chosen from the group consisting of uncrosslinked PVA with a molecular weight of 15,000 to 400,000 g/mole, crosslinked PVA and mixtures thereof. The medicotechnical PVA product according to the invention can advantageously be provided in the form of a film, membrane, solution, foam, gel, spray or powder. As a result of its excellent biocompatibility, PVA is particularly suitable for use in the body of a patient. For example, PVA is not modified in vivo, does not lead to inflammatory reactions and is only accumulated to a limited extent in body organs. In principle, PVA is a biodegradable, synthetic polymer with a carbon-carbon backbone. An enzymatic degradation mechanism is known, which lasts several days. The hydroxyl group is oxidized to a keto group and hydrolyzed into methyl ketones and carboxylic acids, accompanied by the cleaving of the carbon-carbon bond.

The excretion of PVA without degradation largely takes place through the kidneys, the excretion rate being dependent on the molecular weight. PVA with a molecular weight of less than 15,000 g/mole is excreted from the body within a few hours, which is too fast for the intended use in adhesion prophylaxis.

With particular advantage the PVA used according to the invention can have a molecular weight of 20,000 to 400,000 g/mole. In one embodiment the PVA can be formed from a mixture of low molecular weight and high molecular weight components and in particular the high molecular weight component is PVA.

Due to its hydroxyl groups polyvinyl alcohol is soluble in water. The solubility and other chemical and physical properties of the polymer can be modified. The modification of PVA can be in the form of a chemical modification, physical modification or a combination of modifications. Examples for a chemical modification are copolymerization, grafting and chemical crosslinking. An example for physical modification is physical crosslinking, the formation of oriented molecular structures, hydrogels and crystallite formation.

A chemical crosslinkling of the crude PVA can generally take place via the alcoholic groups in an addition reaction with diisocyanate or in a condensation reaction with a polyfunctional acid. A reverse insolubility by crosslinking is preferred for biological PVA excretion or elimination. Of particular significance for the provision of the product according to the invention for medical purposes is that no toxic or harmful substances are formed in the body of the patient under physiological conditions. Thus, the invention gives preference to a hydrolyzable crosslinking, particularly by means of polyvalent carboxylic acids, e.g. in the form of anhydrides. However, an enzymatically cleavable crosslinking can also be used. Crosslinking can take place with a metabolizable diisocyanate, which carries a cleavable bond, such as e.g. an ester bond. Thus, no urethane bridges can arise, which are not degraded in the body or are only degraded after a long time.

In an embodiment of the invention PVA with a molecular weight of 15,000 to 400,000 g/mole can be chemically crosslinked. Advantageously the product according to the invention is characterized in that crosslinking takes place by means of crosslinking agents, which give an in vivo reversible crosslinking, particularly a crosslinking reversible by chemical hydrolysis. In the case of resorbable polymers the crosslinking points are preferably chemically hydrolyzable and not enzymatically cleavable. In a preferred embodiment, in connection with the product according to the invention chemical crosslinking can take place by esterification for adhesion prophylaxis purposes.

According to the invention preferred crosslinking agents are polyvalent dicarboxylic acids and/or their derivatives. The esterification of alcoholic groups in the PVA with dicarboxylic acids is characterized as a reversible crosslinking reaction. In particular, anhydrides of carboxylic acids can be used as crosslinking agents. Examples of such crosslinking agents are succinic anhydride or oxalic anhydride. Succinic anhydride is more reactive than succinic acid, because ring opening energy becomes free.

Other crosslinking agents with at least two functional groups are also possible. The crosslinking agent can also be constituted by compounds with double bonds and imides and acrylates are examples thereof.

If the crosslinking reaction is performed in solution, a shorter chain crosslinking agent is more advantageous, because there is a limited probability of an intramolecular reaction with only a single polymer chain. Thus, the invention gives preference to oxalic acid and its derivatives due to the shorter molecular chain.

In another embodiment of the invention PVA with a molecular weight of 15,000 to 400,000 g/mole can be physically crosslinked. Advantageously the physical crosslinking can be carried out by crystallite formation. In such a physical crosslinking a three-dimensional network of PVA molecules is formed, which are held together by crystallites as physical crosslinkling points.

For effecting a physical crosslinking, an aqueous solution of PVA can be frozen at −20° C. for 6 to 48 hours and then thawed at 25° C. for 2 to 6 hours, so that the desired crosslinking occurs. According to the invention the preparation of the PVA hydrogels can take place by means of a freezing/thawing cycle, which advantageously is repeated several times. In the case of physical crosslinking by freezing/thawing cycles, different phases can be associated with different temperatures. In particular, nanoparticles can be produced by freezing/thawing cycles.

The PVA chains can also be modified, in that only a few PVA hydroxyl groups are connected to additional radicals, preferably via ester and/or ether groups. Fatty acids and/or alcohols with a chain length of C₂ to C₁₆ are particularly suitable for such a modification. It is also possible to link amino acids or peptides. It is sufficient to have 1 to 10, particularly 1 to 2 radicals per PVA molecule. Through the modification gelling results from a PVA solution on heating to body temperature. If e.g. a PVA solution is applied at room temperature, e.g. by spraying, on heating to body temperature (37° C.) immediately a film is formed, which brings about the desired adhesion prophylaxis.

In another embodiment PVA can be present in a mixture with a high molecular weight component, which is not PVA. Such a high molecular weight component can be present in a quantity of 0.5 to 4 wt. %, particularly 1 to 2 wt. %. The high molecular weight component can be applied as a bilayer to the PVA. Such a layer structure can e.g. be formed by spraying the high molecular weight component onto a PVA membrane. The high molecular weight component not formed from PVA can also be provided for additional medicament absorption.

Advantageously a sugar polymer is added as the high molecular weight component to the PVA according to the invention. The sugar polymer can in particular be chosen from the group consisting of carboxymethyl cellulose, dextran, hydroxymethyl cellulose, hydroxyethyl starch, chitin and/or heparin.

According to an embodiment of the present invention the product for adhesion prophylaxis can be present in the form of an at least one-layer film. PVA films can be produced industrially by extrusion. In particular, PVA films can be produced by a casting process. In particular, thin and homogeneous membranes with a thickness of approximately 5 to 7 μm can be produced by the spin casting process.

According to the invention the film can be formed from a single layer only. In another embodiment of the invention the film can be formed from two layers, namely a so-called bilayer. Examples for such a bilayer are two layers based on PVA or a combination of PVA and a further component selected from the group of carbohydrates, lipids and proteins, as well as derivatives thereof. Preference is given to a bilayer of PVA and CMC (carboxymethyl cellulose). In another embodiment of the invention the film can comprise three layers, a so-called trilayer. Examples are sandwich-like combinations of PVA and CMC, such as the layer sequence CMC/PVA/CMC, PVA/CMC/PVA, CMC/PVA/PVA or CMC/CMC/PVA, as well as combinations of PVA, CMC and a further component selected from the group of carbohydrates, lipids and proteins, together with their derivatives.

In sandwich-like structures the layers formed from PVA, CMC and optionally other components can be interconnected, e.g. by bonding or welding. The possible adhesives are water, aqueous solutions of PVA or CMC. Welding can take place by the application of heat, preferably in the form of spot welding, or ultrasonics.

If the second layer connected to the PVA layer is in the form of an open-pore material, e.g. in the preferred form of a fleece more particularly obtained by lyophilization, then there is no need for special fastening of the sandwich or bilayer structure to the tissue of the patient, because the open structure brings about a self-adhesion.

A layer-like product for adhesion prophylaxis can be in unmoulded form. In another embodiment a layer-like product for adhesion prophylaxis can be in moulded form. As a result of moulding it is in particular possible to apply a surface structuring, e.g. embossing.

According to the invention, on at least one side, the film can have a structuring. Such a structure can e.g. be formed by casting on a structured surface or by embossing. Preferably there is a structuring in geometrical shapes. Such a surface structure can be differently designed on the two sides of the layer-like product.

According to another embodiment of the invention, the adhesion prophylaxis product can be in the form of a foam or a foam precursor. In a particular embodiment at least one layer can be provided in the form of a foam or a foam precursor.

According to another embodiment of the invention the adhesion prophylaxis product can be in the form of a solution, which is preferably sprayed.

According to yet another embodiment of the invention the adhesion prophylaxis product can be in the form of a gel, more especially a microgel. Preferably it is in the form of a dimensionally stable hydrogel. Microgels can be sprayed with suitable propellant gases, so that in this way they can be used as spray gels for adhesion prophylaxis.

The PVA according to the invention is in particularly preferred manner present in the form of microparticles with diameters in the nanometre range, so-called nanoparticles. The nanoparticles can be provided in different forms as a medicotechnical product. Examples are the aforementioned application forms such as gels, films or sprays. It is also possible to provide on a film surface microparticles and in particular nanoparticles.

The medicotechnical product according to the invention is advantageously characterized in that it is in a form swollen with aqueous media. With weakly crosslinked, dry polymers swelling occurs in aqueous media or suitable solvents, the swelling starting at the surface and propagates into the interior. The swelling rate is not influenced by the diffusion coefficient of the swelling agent, but instead by the diffusion rate of segments of the polymer. The more the swelling advances, the more noticeable the elastic restoring forces of the crosslinked polymer chains. Gels have viscoelastic characteristics. Swelling takes place up to a maximum value. Unlike in the case of uncrosslinked polymers, which can be prepared from aqueous solutions in variable concentration, chemically crosslinked polymers are not dissolved.

The adhesion prophylaxis product according to the invention can in the form of a membrane dried in the laminar flow, by swelling, absorb a liquid quantity, such as e.g. water, representing 20% of the product weight. Due to the absorption of liquid during swelling a gel is formed, which exerts friction-inhibiting functions. The adhesion prophylaxis product can be 80% water in the form of a hydrogel. For the application according to the invention in adhesion prophylaxis it can consequently take over the mesothelium function.

For use in adhesion prophylaxis it is desirable for both membranes and gels not to be too rigid. The rigidity of the gels results from their modulus of elasticity, which is directly proportional to the concentration of elastic network chains. Covalently crosslinked gels have a size of d=3. The modulus of elasticity increases with rising concentration according to the formula E=kc^{9/4}.

Covalently and physically crosslinked gels differ in the concentration and polymerization degree of the moduli. In principle, physical networks can be seen in the same way as a plate of spaghetti and hooking together arises. In both cases the modulus is controlled by the concentration at the network points. With covalently linked networks only the degree of swelling and consequently the concentration of the crosslinking agent in the gel is responsible. The degree of polymerization is infinitely high and can also not be changed by shearing. This plays a major part in normal body movements, because the degree of polymerization remains unchanged. In covalent networks no sliding of the chains is possible. In physical networks, accompanied by shearing, the network points are broken and reconnected again. An only physically crosslinked polymer can under certain conditions be washed off the surface again and is therefore suitable in surgery for a limited residence time and limited period of operation in the body. A particular advantage of covalently crosslinked polymers is that their characteristics can be adjusted as desired.

Polyvinyl alcohol in the form of a hydrogel is a rubbery material and its elasticity can be so adjusted via clearly defined network points that it is very close to soft body tissue or muscles. Simultaneously PVA has a high tensile strength.

According to the invention the molecular weight of the PVA or the mixture can be chosen in such a way that it can be excreted via the kidneys essentially without degradation of the PVA molecules. If desired, following hydrolysis or an elimination of the crosslinking, PVA can be excreted via the kidneys essentially without any degradation of the PVA molecules.

According to the invention the degree of crosslinking of the medicotechnical PVA product can be adjusted in such a way that its period of operation is 5 to 21 and in particular 5 to 14 days. For adhesion prophylaxis it is desirable for the material to remain for at least 5 days in the peritoneal cavity. A control of the excretion time can take place through a setting of parameters influencing PVA excretion. Influencing factors are e.g. chemical crosslinking, physical crosslinking of the PVA material, layer thickness, mixture composition and the addition of additives such as sugar polymers.

Advantageously the macroscopic dissolving of the medicotechnical product according to the invention can be 7 to 60 days under physiological conditions. The residence time of the PVA in the body is also dependent on the hydrodynamic radius of the polymer molecules. According to the invention PVA advantageously has a hydrodynamic radius of 5 to 15 nm, particularly 5 nm.

With particular advantage the crosslinked PVA can have a wetting behaviour advantageous in a physiological environment. The medicotechnical adhesion prophylaxis product according to the invention can in particular be characterized in that the crosslinked PVA has a structure allowing in a physiological medium an exchange of material of small molecules, but essentially prevents a deposition of physiological substances, such as in particular blood and cells.

For this purpose structural characteristics of the medicotechnical product such as the surface structure, pore structure and network density of a PVA membrane can be adapted in such a way that it is permeable for small molecules such as e.g. water, glucose or nutrients, but prevents the deposition of large particles, such as e.g. blood, body cells, macrophages or microorganisms, such as e.g. pathogenic bacteria.

PVA solutions and blends can have a LCST (lower critical solution temperature) behaviour. Below this critical temperature the PVA is in dissolved form. Above this temperature the PVA polymer precipitates. The phase behaviour can be controlled by modifications. Examples of such modifications are the binding of short radicals with a chain length of C₂ to C₁₆, e.g. dodecyl radicals or short-chain fatty acids, carbohydrates, amino acids, peptides or blends, i.e. mixing with other polymers. In this way a PVA solution can immediately form a film at room temperature following application to the patient, e.g. spraying on body tissue at the body temperature of 37° C.

The present invention also relates to a method for the manufacture of a medicotechnical product for adhesion prophylaxis, which is characterized in that it is formed from at least one PVA selected from the group consisting of uncrosslinked PVA with a molecular weight of 15,000 to 400,000 g/mole, crosslinked PVA and mixtures thereof, the molecular weight of the PVA or the mixture being selected in such a way that, optionally following the elimination of the crosslinking, it can be substantially excreted via the kidneys without any degradation of the PVA molecules.

With particular advantage the medicotechnical product is lyophilized. As a result of lyophilization the PVA according to the invention can be transformed into a spongy structure. Such a structure improves the handling characteristics of the medicotechnical product.

In an embodiment of the invention PVA can be physically crosslinked, particularly by crystallite formation. Advantageously the physical crosslinking is performed by freezing/thawing cycles, which are in particular repeated several times. Preferably nanoparticles can be produced by the freezing/thawing cycles.

For producing PVA particles a water-in-oil emulsion is homogenized and said emulsion is frozen. No surfactant addition is necessary. The size of the resulting particles depends on the duration and the set speed (revolutions per minute) in the homogenizer and naturally the PVA concentration. The size of the PVA particles can be adjusted from nanometres, through micrometres to millimetres. The number of freezing/thawing cycles determines the degree of crystallinity and therefore the swelling behaviour. Following the freezing cycles said PVA nanoparticles or microparticles can be separated by filtration and dried. Such particles can e.g. be sprayed in spray form. Advantageously they can in this way be used in adhesion prophylaxis.

In another embodiment of the invention PVA can be chemically crosslinked and in particular crosslinked in a solvent mixture. Preference can be given to crosslinking reversible under physiological conditions. Preference is given to a chemical crosslinking by means of polyvalent carboxylic acids, particularly their derivatives.

According to the method of the invention, the dissolving behaviour, particularly the period of operation of PVA, preferably uncrosslinked PVA or physically crosslinked PVA, with a molecular weight in the range 15,000 to 400,000 g/mole, can be adjusted to the desired level by mixing with high molecular weight components, particularly PVA and/or sugar polymers.

Advantageously crosslinking can be carried out to a desired level. According to the invention the dissolving behaviour, particularly the period of operation, can be adjusted by the degree of crosslinking.

Preference is given according to the invention to esterification reactions, which take place in acid-catalyzed and therefore pH-dependent manner. An important process parameter is the PVA concentration in the aqueous reaction solution. For example, for a PVA with a molecular weight of 22,000 g/mole, a concentration in aqueous solution of at least 6 wt. % is necessary. The higher the temperature, the faster the condensation reaction takes place. The gel formation time is dependent on the viscosity rise. The gel formation time decreases by half if the temperature rises by 10° C. The gel formation time rises by several hours if the pH is increased, e.g. from 9 to 42 hours with a pH change from 1.2 to 2.9.

In an embodiment the crosslinking reaction can be performed on prefabricated products, particularly PVA films. Homogeneous films can be produced by spin casting from PVA in aqueous solution. If such films are brought after drying into a solvent mixture of 90% acetone and 10% water, the water can swell the PVA membrane without dissolving it. A chemical crosslinking agent can easily penetrate a PVA membrane preswollen in this way and can be washed out again when the intended reaction has taken place.

The invention also relates to the provision of the medicotechnical product in the manner described hereinbefore for use in the prophylaxis of adhesions during surgery in human and veterinary medicine. The PVA-based product according to the invention can, as a function of the medical requirements and the desired manner of use, be provided or prepared for use in different forms. It can e.g. be in the form of a membrane, solution, foam, gel or spray. The product can be wholly or partly dyed. The medicotechnical product according to the invention can be provided as a coating on surgical materials, such as e.g. implants.

Advantageously the medicotechnical product is provided in the form of a membrane for the physical separation of tissue layers in the body.

In another embodiment the medicotechnical product in the form of a multilayer membrane or film can be used for the physical separation of tissue layers in the body and preferably one side is constructed in such a way that tissue adhesion is avoided and the other is constructed in such a way, particularly through the structuring of the surface, that tissue adhesion is aided. Thus, during surgery, there is no need for sewing the film or membrane for the purpose of fixing in the body.

In another embodiment the medicotechnical product in the form of a solution can be used for the physical separation of tissue layers in the body.

In another embodiment the medicotechnical product in the form of a film can be used for the physical separation of tissue layers in the body.

In yet another embodiment the medicotechnical product in the form of a gel, especially a spray gel, can be used for the physical separation of tissue layers in the body.

In another embodiment the medicotechnical product in the form of nanoparticles can be intended for the physical separation of tissue layers.

In another embodiment the medicotechnical product in the form of a spray can be used for the physical separation of tissue layers.

In a special embodiment the medicotechnical product combined with a separate, biocompatible component can be used for the physical separation of tissue layers. An example is the interaction of PVA with ionic components, whereby during the combination thereof in the field of operation a gel is formed via ion bridges and is used for adhesion prophylaxis.

Further features and details of the invention can be gathered from the following description of preferred embodiments in the form of examples. The individual features can be implemented singly or in combination with one another. The examples merely serve to illustrate the present invention, which is in no way restricted thereto.

Production of Membranes and Hydrogels from PVA

### EXAMPLE 1

### Casting Process

PVA is weighed into a screw fastenable Schott glass, topped up with deionized water and dissolved in the drying oven at 90° C. In this way the process is very rapid and frothing can be avoided (degassing being unnecessary). The solutions are filtered by means of a disposable syringe through sterile filters (0.45 μm pore diameter), poured onto Teflon and dried in the laminar flow. The desired membrane thickness can be controlled with the filled quantity. Most suitable are aqueous 4 to 20% PVA solutions. The membranes can be cut to the desired size.

If a structured surface is desired, a selected structure can be preshaped in the Teflon and is then transferred to the product.

### EXAMPLE 2

### PVA Viscosity

The decisive parameter for the processing of dissolved, high molecular weight substances is the viscosity. It is possible to filter 8 to 10% PVA solutions, which can also be easily handled. The filtration of solutions using the 0.45 μm filter is possible up to a viscosity of 25 mPas, but larger pore diameter filters must be used for higher viscosities. Pouring or casting is completely unproblematical up to 50 mPas, whereas for higher viscosities very slow casting should occur and possibly occurring air bubbles should be perforated with a needle or transferred to the edge by a slide drawn over the surface.

### EXAMPLE 3

### Membrane Characteristics

The membranes are smooth and transparent but, if desired, can absorb the substrate structure and therefore appear milky. Through conditioning (i.e. water absorption) in the air conditioning cabinet, this can be eliminated again or adjusted. The membranes are homogeneous and reveal no pores even in the case of a 1500×magnification. There are to be no holes in the REM and to make the image sharp it is necessary to focus dust particles or the like.

### EXAMPLE 4

### Membrane Conditioning

The PVA membranes are relatively brittle due to the long drying phases in air. A conditioning under clearly defined heat and moisture conditions is indispensable. The air conditioning cabinet is set to 37° C. and 100% atmospheric humidity. Under the action of moisture, the membranes very rapidly become soft and flexible. To find optimum conditioning circumstances, from different sample compositions are cut test strips (4×1 cm) and stored for different time periods in the air conditioning cabinet. Investigations regarding the mechanical characteristics have revealed that within the first three days in the cabinet the breaking force decreases. After about 7 days the membranes are saturated and the breaking force passes to a plateau value. The breaking force is then about half the initial value. Error averaging with in each case 20 samples from three batches, after three days conditioning gives an error range of 10 N/mm² (delta=10 N/mm²). The expansion of the samples clearly increases with water absorption. The error range after three days in the air conditioning cabinet is 30% (delta=30%). Conditioning increases the expansion of the samples to 500%, i.e. in the relevant time period the membranes withstand without tearing the movements and associated expansions resulting from normal body and intestinal movements. After storing for only one day in the air conditioning cabinet the expansibility of the films increases to more than double and a plateau value (such as is also the case when measuring the breaking force) is found after about 6 to 7 days.

### EXAMPLE 5

### Additives

An admixing of a few per cent high molecular weight PVA is clearly expressed in the expansion measurements. The addition of only 0.5 to 1 wt. % high molecular weight PVA increases the expansibility of the membranes by up to 100%. An increase can consequently take place by mixing different PVAs with different molecular weights.

### EXAMPLE 6

### Resorption Behaviour

The resorbability of membranes with different thicknesses (from 0.06 to 0.16 mm) was investigated at 37° C. in an aqueous solution. The time-dependent dissolving of the different membranes can be controlled by varying the composition and thickness. Membranes made from relatively short PVA (mol. wt.=20,000 g/mole) are stable as from a thickness of 0.12 mm over a period of 4 weeks. Thinner films dissolve rapidly and initially disintegrate into larger fragments unable to offer a complete protection against adhesions. For homologous mixtures, i.e. blends of PVA with a high molecular weight component, membranes with a thickness of ≧0.1 mm are stable over a period of 4 weeks. Both the pure PVA films and also blends appear transparent in solution.

### EXAMPLE 7

### Influence of Additives on Resorption

On admixing carboxymethyl cellulose (CMC for short) or other carbohydrates, there is a change to the resorption rate and the disintegration mechanism. The films swell more and appear turbid, but do not disintegrate into larger fragments. On the membrane surface smaller holes, which increase in size are formed. The addition of 0.25 wt. % CMC already changes the disintegration behaviour of the membranes.

### EXAMPLE 8

### Sterilization

Gamma radiation, EtOx sterilization and plasma sterilization are examined as sterilization methods. A risk arising during sterilization is that radicals can form in the material and can lead to stable carbon-carbon linkages within the membrane. A consequence thereof could be an insolubility of the material and therefore a completely changed resorption. Such new bonds in the material have a dramatic effect on the viscosity. Thus for investigation purposes the sterilized membranes were redissolved and measured in the viscotester. The viscosities of the membranes following gamma sterilization are below the initial values and this can be explained by the fact that the films contain water and therefore to some extent falsify the weighed portion. The Co60 radiation was in the range 26.2 to 30.4 kGy. Plasma sterilization also involves drying stages in vacuo and consequently there is here a very good coincidence of the viscosity with the 8% starting solution. In no case is crosslinking detected (checking several batches) All the sterilization samples were sterile, i.e. all three methods can be used.

### EXAMPLE 9

### Biocompatibility

The biocompatibility of the membranes tested is excellent, e.g. the acute systemic toxicity test in mice revealed no difference compared with the injected control solutions. The injections were partly made intravenously and partly intraperitoneally. The animals were observed 4, 24, 48 and 72 hours after injection. The same applies e.g. for cytotoxicity tests with mouse fibroblasts, which also revealed good results. No change to the cytomorphology or indication of toxic actions were discovered. The material is no cytotoxic and has no acute systemic toxicity.

### EXAMPLE 10

### Hydrogels

PVA hydrogels can be produced by freezing-thawing processes. 20% PVA solutions are frozen in a Petri dish at −20° C. This process can be performed in a number of cycles, freezing for 12 hours and thawing again for 2 hours in the covered state. The resulting PVA hydrogels are dimensionally stable and adhesive. The higher the PVA molecular weight, the more dimensionally stable the resulting gel.

### EXAMPLE 11

### Nanoparticles

Based on the freezing-thawing cycles of example 10, PVA particles can also be produced. PVA or PVA mixtures (different molecular weights) are dissolved in water or phosphate buffer at pH 7.4. The aqueous solution in a volume proportion of 2 to 20% is brought into a large silicone oil excess. This mixture is homogenized in a homogenizer for 5 minutes at 10,000 revolutions per minute to give a water-in-oil emulsion. Following 1 to 10 freezing cycles at −20° C., the PVA particles are extracted with acetone in a volume ratio of 1:10. The PVA particles are filtered off, washed with acetone and dried in vacuo. The nanoparticles are almost round, with a rough surface. PVA hydrogel nanoparticles on average have a size 680±40 nm.

### EXAMPLE 12

### Improvement of Tissue Adhesion

Research was carried out concerning tissue adhesion of PVA membranes dried in the laminar flow or dimensionally stable PVA hydrogels produced by freezing/thawing cycles.

The adhesion behaviour of PVA products can be improved by surface roughening. This can be brought about by casting PVA on substrates having clearly defined surfaces. A corrugated substrate can be used for this purpose. Interruptions can subsequently be made in the PVA surface, e.g. using needles in the micrometre range or by moulding in a pattern.

Tissue adhesion can also be improved by crosslinking gradient formation. This can be achieved by setting different temperatures during gel formation.

Adhesion can also be improved by chemical modification of the PVA. The hydrophilicity of the membrane surface can be increased by introducing functional groups such as carboxyl groups. The hydrophilicity of the PVA membrane can also be influenced by the nature of the substrate on which it is cast. Thus, casting on polar surfaces such as glass increases hydrophilicity.

In the case of PVA hydrogels produced by freezing-thawing cycles, a spongy structure can be produced by lyophilization in the lyophilizer. Such a spongy structure improves handling and adhesion on moist tissue without impairing effectiveness. Through further processing operations such as moulding, the behaviour of the spongy structure can be adapted to the tissue.

PVA membranes produced by solvent evaporation can be sprayed with PVA solution and then lyophilized. The double layer formed has the advantage of a complete membrane on the one hand and a bioadhesive spongy structure on the other.

PVA hydrogels produced by freezing-thawing cycles can be exposed temperature gradient, so that bioadhesive characteristics are formed in the material. The advantageous characteristics result from the different density of the physical network points due to the gradient.

PVA solutions can be sprayed using nozzles and in this way processed to a fleece, which is used with advantage in adhesion prophylaxis.

### EXAMPLE 13

### PVA Coating

In surgery polypropylene textile networks are used as implants and very frequently come into direct contact with the visceral abdomen and accretions can occur when the body tissue is traumatized. A coating of the prefabricated PP network with an anti-adhesive PVA coating according to the invention can counteract such accretions. Up to the healing of the tissue after about 7 days, the layer can remain in place and can then be resorbed.

The coating of the networks takes place by immersions in a PVA solution. The coating thickness on the PP network can be controlled by the concentration of the PVA solution or the immersion time.

A very thin coating in the nanometre range can be obtained by immersing the network in a 1% PVA solution at approximately 80° C. No decisive part is played by the PVA molecular weight in connection with the coating process. However, the resorption time in the body is directly linked with the molecular weight. It is therefore preferable for very thin coatings to use high molecular weight PVA with an approximate molecular weight of 200,000 g/mole, so that the coating does not immediately dissolve again and can consequently offer no protection against adhesions. A very thin coating does not change the material characteristics of the network and the PVA layer is transparent.

For a thicker layer in the micrometre range the network to be coated is immersed one or more times in a 3 to 10% PVA solution and frozen. In 1 to 5 freezing-thawing cycles, as a function of the desired strength, in this way a PVA hydrogel is formed. The PVA hydrogel is elastic and does not alter the material characteristics of the network, even in the thicker layer. The resorption time is decisively extended by this physical crosslinking. The higher the molecular weight, the more stable the hydrogel. For the precise setting of the resorption time it is also possible to use a mixture of low molecular weight/high molecular weight PVA. The PVA layer is transparent.

Result of Prophylaxis

Use in Surgery in Animal Tests

### EXAMPLE 14

The caecum of hares is used as the animal model. The surface of the caecum is abraided with a gauze dressing for 15 min. The visceral peritoneum facing the remaining intestinal convolution was abraided for 5 minutes. Testing took place of the percentage of the accretion surface between the wound surface in the parietal peritoneum and the viscal peritoneum on the caecum surface, the tenacity of the accretion and the histological state in the vicinity of the abdominal wall defect.

For adhesion prophylaxis use was made of a PVA film from an 8% solution dried in the laminar flow and having a thickness of 0.09 to 0.1 mm (AAF1), a PVA hydrogel of PVA 1/PVA 2 in a weight ratio of 60:40 in a thickness of 0.15 mm produced by freezing-thawing cycles and a commercial anti-adhesion film as a reference.

The hydrogel according to the invention with PVA 1 comprises relatively short-chain PVA with a molecular weight of approximately 20,000 g/mole and with PVA 2 relatively long chain PVA with a molecular weight of approximately 200,000 g/mole. The hydrogel was produced from a 20% aqueous solution whilst performing the freezing-thawing processes three times. The dimensionally stable hydrogel comprises approximately 80% water.

The prophylactically untreated control group of 12 animals revealed adhesion up to 100% overall surface size. AAF1 and AA2 samples revealed a better effectiveness than the known anti-adhesion film.

In the case of prophylaxis with AAF1 8 of the 12 animals are completely adhesion-free, whilst 4 animals reveal adhesions with an overall surface size of 50, 15, 2 and 10%. In the case of prophylaxis with AAF2 6 of 12 animals have adhesions with a limited overall surface size of 3 to 10%. A statistical analysis of the data in one way analysis of variance test (ANOVA) shows a significant difference compared with the control group. Compared with the known anti-adhesion film, AAF1 and AAF2 reduce the adhesion surfaces. AAF1 and AAF2 do not differ significantly from one another, but the differences compared with the known film are significant.

In the case of animal groups treated with AAF1 and AAF2, to the extent that they occur at all the accretions are delicate and easily dissolvable connective tissue structures. Thus, the PVA films are effective for adhesion prophylaxis.

### EXAMPLE 15

The animal model used was the uterus cornu of hares, which has a long established adhesion system.

The serosa on the uterus cornu was scraped to a length of 5 cm using a fresh scalpel. Thus, punctiform bleeding was produced. In all, operations took place on 12 hares. 8 animals were prophylactically treated with AAF2 and 4 animals were used as the control group. All the animals maintained their weight or slightly increased the same, revealing no sign of acute toxicity. 7 of the 8 animals treated with adhesion prophylaxis revealed no adhesion, whereas one showed slight accretions over roughly 25% of the surface area. A clear adhesion reduction is brought about by PVA adhesion prophylaxis.

1. Medicotechnical product for adhesion prophylaxis for the post-operative prevention of accretions in the body comprising at least one PVA (polyvinyl alcohol) selected from the group comprising uncrosslinked PVA with a molecular weight of 15,000 to 400,000, crosslinked PVA and mixtures thereof.

2. Product according to claim 1, characterized in that the PVA has a molecular weight of 20,000 to 400,000 g/mole.

3. Product according to claim 1 or 2, characterized in that the PVA is formed from a mixture of low and high molecular weight components, whereof at least one is PVA, the high molecular weight component more particularly being high molecular weight PVA.

4. Product according to one of the preceding claims, characterized in that PVA with a molecular weight of 15,000 to 400,000 is chemically crosslinked.

5. Product according to claim 4, characterized in that chemical crosslinking is performed by crosslinking esterification.

6. Product according to claim 4 or 5, characterized in that the chemical crosslinking is carried out using crosslinking agents, which give a crosslinking reversible in vivo and in particular a crosslinking reversible by chemical hydrolysis.

7. Product according to one of the preceding claims, characterized in that as crosslinking agents are provided polyvalent carboxylic acids and/or their derivatives.

8. Product according to one of the preceding claims, characterized in that PVA with a molecular weight of 15,000 to 400,000 is physically crosslinked.

9. Product according to claim 8, characterized in that physical crosslinking is performed by crystallite formation.

10. Product according to one of the preceding claims, characterized in that PVA is modified by radicals bound via hydroxyl groups.

11. Product according to claim 10, characterized in that 1 to 10, particularly 1 to 2 radicals are present per PVA molecule.

12. Product according to claim 10 or 11, characterized in that the C₂ to C₁₆ radicals contain carbon atoms and are in particular carbohydrate, fatty acid and/or alcohol radicals.

13. Product according to one of the preceding claims, characterized in that PVA is mixed with a high molecular weight component, which is not PVA.

14. Product according to claim 13, characterized in that the high molecular weight component is present in a quantity of 0.5 to 4 wt. %, particularly 1 to 2 wt. %.

15. Product according to claim 13 or 14, characterized in that a sugar polymer is added as the high molecular weight component to the PVA.

16. Product according to claim 15, characterized in that the sugar polymer is chosen from the group comprising carboxymethyl cellulose, dextran and/or hydroxymethyl cellulose.

17. Product according to one of the preceding claims, characterized in that it is in the form of an at least one-layer film.

18. Product according to claim 17, characterized in that the film is in the form of a bilayer or trilayer, particularly from PVA and carboxymethyl cellulose.

19. Product according to one of the preceding claims, characterized in that the film has a structuring on at least one side.

20. Product according to one of the preceding claims, characterized in that there is in particular at least one layer in the form of a foam or a foam precursor.

21. Product according to one of the preceding claims, characterized in that it is in the form of a solution.

22. Product according to one of the preceding claims, characterized in that it is in the form of a gel, particularly a microgel.

23. Product according to one of the preceding claims, characterized in that it is in the form of a dimensionally stable hydrogel.

24. Product according to one of the preceding claims, characterized in that it is the form of microparticles, particularly nanoparticles.

25. Product according to one of the preceding claims, characterized in that it is in a form swollen with aqueous media.

26. Product according to one of the preceding claims, characterized in that a liquid quantity of up to 20% of the product weight is absorbed by swelling in a dry membrane.

27. Product according to one of the preceding claims, characterized in that the molecular weight of the PVA or the mixture is chosen in such a way that optionally following a hydrolysis or the elimination of the crosslinking, the PVA molecules are excreted via the kidneys, substantially without degradation.

28. Product according to one of the preceding claims, characterized in that its functioning period in the operating region is 5 to 21 days, particularly 5 to 14 days.

29. Product according to one of the preceding claims, characterized in that its macroscopic dissolving under physiological conditions is 7 to 60 days.

30. Process for the manufacture of a medicotechnical product for adhesion prophylaxis, characterized in that it comprises at least one PVA (polyvinyl alcohol) selected from the group consisting of uncrosslinked PVA with a molecular weight of 15,000 to 400,000, crosslinked PVA and mixtures thereof, the molecular weight of the PVA or the mixture being chosen in such a way that, optionally after eliminating the crosslinking, it is excreted via the kidneys substantially without degradation of the PVA molecules.

31. Process according to claim 30, characterized in that the medicotechnical product is lyophilized.

32. Process according to claim 30 or 31, characterized in that PVA is physically crosslinked, particularly by crystallite formation.

33. Process according to one of the claims 30 to 32, characterized in that the physical crosslinking is carried out by freezing-thawing cycles, which are in particular repeated several times.

34. Process according to one of the claims 30 to 33, characterized in that nanoparticles are produced by freezing-thawing cycles.

35. Process according to claim 30 or 31, characterized in that PVA is chemically crosslinked, particularly in a solvent mixture, a crosslinking reversible under physiological conditions being preferred.

36. Process according to claim 30 or 35, characterized in that polyvalent carboxylic acids, particularly their derivatives are used as crosslinking agents.

37. Process according to one of the claims 30 to 36, characterized in that the dissolving behaviour and in particular the functioning period of PVA, preferably uncrosslinked PVA or physically crosslinked PVA, with a molecular weight of 15,000 to 400,000 is set to the desired level by mixing with high molecular weight components, particularly PVA and/or sugar polymers.

38. Process according to one of the claims 30 to 37, characterized in that the dissolving behaviour, particularly the functioning period is set by the degree of crosslinking.

39. Process according to one of the claims 30, 31 or 35 to 38, characterized in that PVA in the form of a prefabricated product, particularly a film or sheet is crosslinked.

40. Provision of the medicotechnical product according to one of the claims 1 to 29 for use in the prophylaxis of adhesions in surgery in human and veterinary medicine.

41. Provision according to claim 40, characterized in that the medicotechnical product in the form of a membrane is intended for the physical separation of tissue layers.

42. Provision according to claim 40, characterized in that the medicotechnical product in the form of a multilayer membrane or film is intended for the physical separation of tissue layers and preferably one side is constructed in such a way that tissue adhesions are avoided and the other is constructed in such a way, particularly through surface structuring, that tissue adhesions are aided.

43. Provision according to claim 40, characterized in that the medicotechnical product in the form of a solution is intended for the physical separation of tissue layers.

44. Provision according to claim 40, characterized in that the medicotechnical product in the form of a foam is intended for the physical separation of tissue layers.

45. Provision according to claim 40, characterized in that the medicotechnical product in the form of a gel, especially a spray gel, is intended for the physical separation of tissue layers.

46. Provision according to claim 40, characterized in that the medicotechnical product in the form of nanoparticles is intended for the physical separation of tissue layers.

47. Provision according to claim 40, characterized in that the medicotechnical product in the form of a spray is intended for the physical separation of tissue layers.
